(19) United States
(12) Patent Application Publication
(10) Pub. No.: US 2021/0262010 A1
(43) Pub. Date: Aug. 26, 2021

(54) METHODS FOR ANALYZING CELLS

(71) Applicant: Coral Genomics, Inc., San Carlos, CA (US)

(72) Inventors: Jacob BORRAJO, Cambridge, MA (US); Atray DIXIT, Cambridge, MA (US)

(21) Appl. No.: 17/122,678

(22) Filed: Dec. 15, 2020

Related U.S. Application Data
(63) Continuation of application No. PCT/US19/41159, filed on Jul. 10, 2019.
(60) Provisional application No. 62/711,444, filed on Jul. 27, 2018.
(60) Provisional application No. 62/697,972, filed on Jul. 13, 2018.

(51) Int. Cl. C12Q 1/6806

(52) U.S. Cl. CPC C12Q 1/6806

(57) ABSTRACT

The present disclosure provides methods for sample processing and analysis. A method of analyzing a plurality of cells may comprise providing a plurality of cells derived from cells of a plurality of subjects, which plurality of cells comprise nucleic acid molecules comprising barcode sequences identifying them as deriving from a subject of the plurality of subjects. Nucleic acid molecules derived from a plurality of nucleic acid molecules of the plurality of cells may be sequenced to provide a plurality of sequencing reads, and the resultant sequencing reads may be processed to associate a subset of the plurality of sequencing reads with a subject.

### CROSS-REFERENCE

This application is a continuation of International Application No. PCT/US19/41159, filed Jul. 10, 2019, which claims the benefit of U.S. Provisional Patent Application Ser. No. 62/697,972, filed Jul. 13, 2018, and U.S. Provisional Patent Application Ser. No. 62/711,444 filed Jul. 27, 2018, each of which is entirely incorporated herein by reference.

### BACKGROUND

Nucleic acid sequencing technologies have dropped the cost of the genome by a factor of >1,000 in the last decade alone. These technological improvements have been achieved by coupling advancements in cameras, sequencing by synthesis, and clonal amplification of deoxyribonucleic acid (DNA) on a substrate. This highly parallelizable approach, named next-generation sequencing (NGS), has powered discoveries and innovations in fields spanning from agriculture to Clustered Regularly Interspaced Short Palindromic Repeats (CRISPR). Such innovations have facilitated genetic analyses and identification of associations between genotypes and phenotypes. However, the complexity and expense of such analyses remains significant.

### SUMMARY

Recognized herein is a need to provide improved methods of analyzing cells and nucleic acid molecules. The methods described herein may facilitate the identification of associations between genotypes and phenotypes within cells and/or subjects from which cells derive. These methods may involve analyzing cells from a plurality of subjects that incorporate representative amounts of genetic diversity. Such methods leverage experimental advances in pooled screening assays and computational sparse inference to increase the throughput and multiplexing capacity of such assays by, in some instances, orders of magnitude. The methods provided herein may allow for a plurality of processes, including, for example, cell derivation, genotyping, perturbation, and phenotyping, to be performed en masse.

In an aspect, the present disclosure provides a method of analyzing a plurality of cells, comprising: (a) providing a plurality of cells derived from cells of a plurality of subjects, wherein the plurality of cells comprise a plurality of nucleic acid molecules, and wherein the plurality of nucleic acid molecules comprise a plurality of barcode sequences; (b) sequencing nucleic acid molecules derived from the plurality of nucleic acid molecules of the plurality of cells, thereby generating a plurality of sequencing reads corresponding to the plurality of nucleic acid molecules, wherein a portion of the plurality of sequencing reads comprise the plurality of barcode sequences; (c) processing the plurality of sequencing reads, which plurality of sequencing reads comprises the plurality barcode sequences; and (d) using a barcode sequence of the plurality of barcode sequences to associate a subset of the plurality of sequencing reads with a subject of the plurality of subjects, wherein, prior to (b), the plurality of cells is generated upon proliferating the cells of the plurality of subjects in a bulk growth environment.

In some embodiments, a subset of the plurality of nucleic acid molecules comprises the plurality of barcode sequences. In some embodiments, the plurality of barcode sequences is endogenous to the plurality of cells. In some embodiments, the method further comprises, prior to (a), incorporating the plurality of barcode sequences into the plurality of nucleic acid molecules of the plurality of cells. In some embodiments, the plurality of barcode sequences is incorporated into the plurality of cells via transduction. In some embodiments, the plurality of barcode sequences is incorporated into the plurality of cells using a viral vector, transfection, homologous recombinant integration, Agrobacterium mediated gene transfer, an antibody-conjugated oligonucleotide, or an episomal vector.

In some embodiments, the barcode sequence of the plurality of barcode sequences comprises from 1 base to 1000 bases. In some embodiments, the plurality of subjects comprises a plurality of human subjects. In some embodiments, the identities of the plurality of subjects are encrypted or ambiguated.

In some embodiments, the plurality of cells is derived from a bodily fluid. In some embodiments, the bodily fluid comprises blood, plasma, urine, sweat, or saliva. In some embodiments, the plurality of cells comprises skin cells or hair cells. In some embodiments, the plurality of cells comprises plant cells. In some embodiments, the plant cells are derived from a leaf or root of a plant.

In some embodiments, proliferated cells of the plurality of cells are stratified by growth rate. In some embodiments, the plurality of cells are stained with carboxyfluorescein succinimidyl ester (CFSE). In some embodiments, at least a subset of the plurality of barcode sequences comprises a plurality of perturbation barcode sequences associated with a plurality of perturbations. In some embodiments, the plurality of perturbations are selected from the group consisting of addition of a small molecule, a knockout, an antibody, cell-cell interactions, RNAi, an open reading frame (ORF), and Clustered Regularly Interspaced Short Palindromic Repeats (CRISPR) single guide ribonucleic acid (sgRNA). In some embodiments, the plurality of perturbations comprise a variation in temperature or a variation in pH. In some embodiments, the plurality of perturbations comprise introduction of mutated forms of genes.

In some embodiments, at least a subset of the plurality of barcode sequences are associated with a plurality of measurements. In some embodiments, the plurality of measurements is selected from the group consisting of RNA-seq, ATAC-seq, in-situ sequencing, and cell morphology measurements. In some embodiments, the method further comprises: (e) introducing a plurality of fluorescent probes to the plurality of cells; (f) subjecting the plurality of cells to conditions sufficient to hybridize the plurality of fluorescent probes to the plurality of barcode sequences; and (g) optically detecting the plurality of fluorescent probes hybridized to the plurality of barcode sequences in the plurality of cells. In some embodiments, the method further comprises, repeating (e)-(g) one or more times. In some embodiments, (c) or (d) comprises use of an external database. In some embodiments, the method further comprises, prior to (b), processing the plurality of nucleic acid molecules to generate the nucleic acid molecules, which nucleic acid molecules are subsequently sequenced. In some embodiments, the processing comprises generating copies of the plurality of nucleic acid molecules. In some embodiments, the processing comprises recovering the plurality of nucleic acid molecules from the plurality of cells.

In another aspect, the present disclosure provides a method of analyzing a plurality of cells, comprising: (a) providing a first plurality of cells derived from cells of a plurality of subjects, wherein the first plurality of cells comprises a first plurality of nucleic acid molecules, and wherein the first plurality of nucleic acid molecules comprises a first plurality of barcode sequences; (b) subjecting the first plurality of cells to conditions sufficient to duplicate cells of the first plurality of cells, to provide a second plurality of cells comprising the cells of the first plurality of cells and duplicates thereof, wherein the second plurality of cells comprises a second plurality of nucleic acid molecules comprising a second plurality of barcode sequences; (c) partitioning cells of the first plurality of cells and the second plurality of cells between a plurality of partitions, thereby providing a plurality of partitioned cells; and (d) sequencing nucleic acid molecules derived from the plurality of partitioned cells, thereby generating a plurality of sequencing reads corresponding to the second plurality of nucleic acid molecules of the plurality of partitioned cells, wherein a portion of the plurality of sequencing reads comprise the second plurality of barcode sequences; (e) processing the plurality of sequencing reads, which plurality of sequencing reads comprises the second plurality of barcode sequences; and (f) using a barcode sequence of the second plurality of barcode sequences to associate a subset of the plurality of sequencing reads with a subject of the plurality of subjects.

In some embodiments, a subset of the first plurality of nucleic acid molecules comprises the first plurality of barcode sequences. In some embodiments, the first plurality of barcode sequences is endogenous to the first plurality of cells.

In some embodiments, the method further comprises, prior to (a), incorporating the first plurality of barcode sequences into the first plurality of nucleic acid molecules of the first plurality of cells. In some embodiments, the first plurality of barcode sequences is incorporated into the first plurality of cells via transduction. In some embodiments, the first plurality of barcode sequences are incorporated into the first plurality of cells using a viral vector, transfection, homologous recombinant integration, Agrobacterium mediated gene transfer, an antibody-conjugated oligonucleotide, or an episomal vector.

In some embodiments, a barcode sequence of the first plurality of barcode sequences or the second plurality of barcode sequences comprises from 1 base to 1000 bases. In some embodiments, the plurality of partitions comprises a plurality of wells. In some embodiments, a well of the plurality of wells comprises one or more cells. In some embodiments, (e) comprises identifying a sequencing read of the plurality of sequencing reads as corresponding to a cell of the plurality of partitioned cells. In some embodiments, the identifying comprises identifying shared sequences of sequencing reads distributed between partitions of the plurality of partitions. In some embodiments, the plurality of partitions comprises a plurality of droplets. In some embodiments, a droplet of the plurality of droplets comprises at most a single cell. In some embodiments, a droplet of the plurality of droplets further comprises a plurality of oligonucleotides, which plurality of oligonucleotides comprise one or more sequencing primers or complements thereof or one or more additional barcode sequences. In some embodiments, (e) comprises identifying a sequencing read of the plurality of sequencing reads as corresponding to a cell of the plurality of partitioned cells.

In some embodiments, the plurality of subjects comprises a plurality of human subjects. In some embodiments, identities of the plurality of subjects are encrypted or ambiguated. In some embodiments, the first plurality of cells is derived from a bodily fluid. In some embodiments, the bodily fluid comprises blood, plasma, urine, sweat, or saliva. In some embodiments, the first plurality of cells comprises skin cells or hair cells. In some embodiments, the first plurality of cells comprises plant cells. In some embodiments, the plant cells are derived from a leaf or root of a plant. In some embodiments, the method further comprises, prior to (d), the first plurality of cells is generated upon proliferating the cells of the plurality of subjects in a bulk growth environment.

In some embodiments, the first plurality of cells and the duplicates thereof are stratified by growth rate. In some embodiments, the first plurality of cells are stained with carboxyfluorescein succinimidyl ester (CFSE). In some embodiments, a portion of the nucleic acid molecules of the plurality of partitioned cells sequenced in (d) comprises a plurality of perturbation barcode sequences associated with a plurality of perturbations. In some embodiments, the plurality of perturbations are selected from the group consisting of addition of a small molecule, a knockout, an antibody, cell-cell interactions, RNAi, an open reading frame (ORF), and Clustered Regularly Interspaced Short Palindromic Repeats (CRISPR) single guide ribonucleic acid (sgRNA). In some embodiments, the plurality of perturbations comprise a variation in temperature or a variation in pH. In some embodiments, the plurality of perturbations comprise introduction of mutated forms of genes.

In some embodiments, a portion of the nucleic acid molecules of the plurality of partitioned cells sequenced in (d) comprises a plurality of barcode sequences associated with a plurality of measurements. In some embodiments, the plurality of measurements are selected from the group consisting of RNA-seq, ATAC-seq, in-situ sequencing, and cell morphology measurements. In some embodiments, the method further comprises: (g) introducing a plurality of fluorescent probes to the first plurality of cells; (h) subjecting the first plurality of cells to conditions sufficient to hybridize the plurality of fluorescent probes to the first plurality of barcode sequences; and (i) optically detecting the plurality of fluorescent probes hybridized to the first plurality of barcode sequences in the first plurality of cells. In some embodiments, the method further comprises repeating (g)-(i) one or more times. In some embodiments, (e) or (f) comprises use of an external database. In some embodiments, the method further comprises, prior to (d), processing the second plurality of nucleic acid molecules to generate the nucleic acid molecules, which nucleic acid molecules are subsequently sequenced. In some embodiments, the processing comprises generating copies of the second plurality of nucleic acid molecules. In some embodiments, the processing comprises recovering the second plurality of nucleic acid molecules from the second plurality of cells.

In another aspect, the present disclosure provides a method of analyzing a plurality of cells, comprising: (a) obtaining a plurality of cells derived from cells of a plurality of subjects; (b) differentially tagging the plurality of cells according to their subject of origin; (c) sequencing nucleic acid molecules derived from a plurality of nucleic acid molecules of the plurality of cells to provide a plurality of sequencing reads; and (d) assigning common sequencing reads of the plurality of sequencing reads to a subject of the plurality of subjects, wherein assigning the common sequencing reads is done independent of variation among the plurality of cells, wherein, prior to (c), the plurality of cells is generated upon proliferating the cells of the plurality of subjects in a bulk growth environment.

In some embodiments, the differentially tagging the plurality of cells comprises introducing a plurality of barcode sequences to the plurality of cells. In some embodiments, the plurality of barcode sequences is incorporated into the plurality of cells via transduction. In some embodiments, the plurality of barcode sequences are incorporated into the plurality of cells using a viral vector, transfection, homologous recombinant integration, Agrobacterium mediated gene transfer, an antibody-conjugated oligonucleotide, or an episomal vector. In some embodiments, a barcode sequence of the plurality of barcode sequences comprises from 1 base to 1000 bases.

In some embodiments, the plurality of subjects comprises a plurality of human subjects. In some embodiments, identities of the plurality of subjects are encrypted or ambiguated. In some embodiments, the plurality of cells is derived from a bodily fluid. In some embodiments, the bodily fluid comprises blood, plasma, urine, sweat, or saliva. In some embodiments, the plurality of cells comprises skin cells or hair cells. In some embodiments, the plurality of cells comprises plant cells. In some embodiments, the plant cells are derived from a leaf or root of a plant.

In some embodiments, the plurality of cells is stratified by growth rate. In some embodiments, the plurality of cells are stained with carboxyfluorescein succinimidyl ester (CFSE). In some embodiments, the plurality of cells sequenced in (c) comprises a plurality of perturbation barcode sequences associated with a plurality of perturbations. In some embodiments, the plurality of perturbations are selected from the group consisting of addition of a small molecule, a knockout, an antibody, cell-cell interactions, RNAi, an open reading frame (ORF), and Clustered Regularly Interspaced Short Palindromic Repeats (CRISPR) single guide ribonucleic acid (sgRNA). In some embodiments, the plurality of perturbations comprise a variation in temperature or a variation in pH. In some embodiments, the plurality of perturbations comprise introduction of mutated forms of genes. In some embodiments, the plurality of cells comprise a plurality of barcode sequences associated with a plurality of measurements. In some embodiments, the plurality of measurements are selected from the group consisting of RNA-seq, ATAC-seq, in-situ sequencing, and cell morphology measurements. In some embodiments, the method further comprises: (e) introducing a plurality of fluorescent probes to the plurality of cells; (f) subjecting the plurality of cells to conditions sufficient to hybridize the plurality of fluorescent probes to the plurality of barcode sequences; and (g) optically detecting the plurality of fluorescent probes hybridized to the plurality of barcode sequences in the plurality of cells. In some embodiments, the method further comprises repeating (e)-(g) one or more times. In some embodiments, (d) comprises use of an external database. In some embodiments, the method further comprises, prior to (c), processing the plurality of nucleic acid molecules to generate the nucleic acid molecules, which nucleic acid molecules are subsequently sequenced. In some embodiments, the processing comprises generating copies of the plurality of nucleic acid molecules. In some embodiments, the processing comprises recovering the plurality of nucleic acid molecules from the plurality of cells.

In another aspect, the present disclosure provides a method of analyzing a plurality of cells, comprising: (a) providing a plurality of cells derived from cells of a plurality of subjects, wherein the plurality of cells comprise a plurality of nucleic acid molecules, and wherein the plurality of nucleic acid molecules comprise a plurality of barcode sequences; (b) sequencing nucleic acid molecules derived from the plurality of nucleic acid molecules of the plurality of cells, thereby generating a plurality of sequencing reads corresponding to the plurality of nucleic acid molecules, wherein a portion of the plurality of sequencing reads comprise the plurality of barcode sequences; (c) processing the plurality of sequencing reads, which plurality of sequencing reads comprises the plurality of barcode sequences; and (d) using a barcode sequence of the plurality of barcode sequences to associate a subset of the plurality of sequencing reads with a subject of the plurality of subjects, wherein the plurality of barcode sequences is incorporated into the plurality of nucleic acid molecules of the plurality of cells via transduction or transfection.

In some embodiments, a subset of the plurality of nucleic acid molecules comprises the plurality of barcode sequences. In some embodiments, the plurality of barcode sequences is endogenous to the plurality of cells. In some embodiments, a barcode sequence of the plurality of barcode sequences comprises from 1 base to 1000 bases. In some embodiments, the plurality of subjects comprises a plurality of human subjects. In some embodiments, identities of the plurality of subjects are encrypted or ambiguated.

In some embodiments, the plurality of cells is derived from a bodily fluid. In some embodiments, the bodily fluid comprises blood, plasma urine, sweat, or saliva. In some embodiments, the plurality of cells comprises skin cells or hair cells. In some embodiments, the plurality of cells comprises plant cells. In some embodiments, the plant cells are derived from a leaf or root of a plant. In some embodiments, prior to (b), the plurality of cells is generated upon proliferating the cells of the plurality of subjects in a bulk growth environment. In some embodiments, proliferated cells of the plurality of cells are stratified by growth rate. In some embodiments, the plurality of cells are stained with carboxyfluorescein succinimidyl ester (CFSE). In some embodiments, the method further comprises: (e) introducing a plurality of fluorescent probes to the plurality of cells; (f) subjecting the plurality of cells to conditions sufficient to hybridize the plurality of fluorescent probes to the plurality of barcode sequences; and (g) optically detecting the plurality of fluorescent probes hybridized to the plurality of barcode sequences in the plurality of cells. In some embodiments, the method further comprises repeating (e)-(g) one or more times. In some embodiments, (c) or (d) comprises use of an external database. In some embodiments, the method further comprises, prior to (b), processing the plurality of nucleic acid molecules to generate the nucleic acid molecules, which nucleic acid molecules are subsequently sequenced. In some embodiments, the processing comprises generating copies of the plurality of nucleic acid molecules. In some embodiments, the processing comprises recovering the plurality of nucleic acid molecules from the plurality of cells.

In another aspect, the present disclosure provides a method of analyzing a plurality of cells, comprising: (a) providing a plurality of cells from a plurality of subjects, wherein the plurality of cells comprise a plurality of nucleic acid molecules, and wherein the plurality of nucleic acid molecules comprise a plurality of barcode sequences; (b) sequencing nucleic acid molecules of the plurality of nucleic acid molecules of the plurality of cells, thereby generating a plurality of sequencing reads corresponding to the plurality of nucleic acid molecules, wherein a portion of the plurality of sequencing reads comprise the plurality of barcode sequences; and (c) processing the plurality of sequencing reads to associate each sequencing read of the plurality of sequencing reads with a given subject of the plurality of subjects.

In some embodiments, the plurality of barcode sequences is subsets of the plurality of nucleic acid molecules.

In some embodiments, the plurality of barcode sequences is endogenous to the plurality of cells.

In some embodiments, the method further comprises, prior to (a), incorporating the plurality of barcode sequences into the first plurality of nucleic acid molecules.

In some embodiments, the plurality of barcode sequences is incorporated into the plurality of cells via transduction. In some embodiments, the plurality of barcode sequences are incorporated into the first plurality of cells using a viral vector, homologous recombinant integration, Agrobacterium mediated gene transfer, or an episomal vector.

In some embodiments, each barcode sequence of the plurality of barcode sequences comprises between 1 and 1000 bases.

In some embodiments, the plurality of subjects comprises a plurality of human subjects. In some embodiments, the identities of the plurality of subjects are encrypted. In some embodiments, the first plurality of cells is derived from a bodily fluid. In some embodiments, the bodily fluid comprises blood, urine, or saliva. In some embodiments, the plurality of cells comprises skin cells or hair cells. In some embodiments, the plurality of cells comprises plant cells. In some embodiments, the plant cells are derived from a leaf or root.

In some embodiments, the plurality of cells is proliferated in a bulk growth environment. In some embodiments, proliferated cells are stratified by growth rate. In some embodiments, the plurality of cells are stained with carboxyfluorescein succinimidyl ester (CFSE).

In another aspect, the present disclosure provides a method of analyzing a plurality of cells, comprising: (a) providing a first plurality of cells from a plurality of subjects, wherein the first plurality of cells comprise a first plurality of nucleic acid molecules, and wherein the first plurality of nucleic acid molecules comprise a plurality of barcode sequences; (b) subjecting the first plurality of cells to conditions sufficient to duplicate cells of the first plurality of cells, to provide a second plurality of cells comprising the cells of first plurality of cells and duplicates thereof, wherein the second plurality of cells comprise a second plurality of nucleic acid molecules comprising the plurality of barcode sequences; (c) partitioning cells of the first plurality of cells and the second plurality of cells between a plurality of partitions, thereby providing a plurality of partitioned cells; (d) sequencing nucleic acid molecules of the plurality of partitioned cells, thereby generating a plurality of sequencing reads corresponding to the plurality of nucleic acid molecules of the plurality of partitioned cells, wherein a portion of the plurality of sequencing reads comprise the plurality of barcode sequences; and (e) processing the plurality of sequencing reads to associate each sequencing read of the plurality of sequencing reads with a given subject of the plurality of subjects.

In some embodiments, the plurality of barcode sequences is subsets of the first plurality of nucleic acid molecules.

In some embodiments, the plurality of barcode sequences is endogenous to the first plurality of cells.

In some embodiments, the method further comprises, prior to (a), incorporating the plurality of barcode sequences into the first plurality of nucleic acid molecules.

In some embodiments, the plurality of barcode sequences is incorporated into the first plurality of cells via transduction. In some embodiments, the plurality of barcode sequences are incorporated into the first plurality of cells using a viral vector, homologous recombinant integration, Agrobacterium mediated gene transfer, or an episomal vector.

In some embodiments, the each barcode sequence of the plurality of barcode sequences comprises between 1 and 1000 bases.

In some embodiments, the plurality of partitions comprises a plurality of wells. In some embodiments, each well of the plurality of wells comprises one or more cells. In some embodiments, (e) comprises identifying each sequencing read of the plurality of sequencing reads as corresponding to a given cell of the plurality of partitioned cells. In some embodiments, the identifying comprises identifying shared sequences of sequencing reads distributed between partitions of the plurality of partitions.

In some embodiments, the plurality of partitions comprises a plurality of droplets. In some embodiments, each droplet of the plurality of droplets comprises one or fewer cells. In some embodiments, each droplet of the plurality of droplets comprises one or more cells. In some embodiments, each droplet of the plurality of droplets further comprises a plurality of oligonucleotides, which plurality of oligonucleotides comprise one or more sequencing primers or complements thereof and/or one or more additional barcode sequences. In some embodiments, (e) comprises identifying each sequencing read of the plurality of sequencing reads as corresponding to a given cell of the plurality of partitioned cells.

In some embodiments, the plurality of subjects comprises a plurality of human subjects. In some embodiments, the identities of the plurality of subjects are encrypted. In some embodiments, the first plurality of cells is derived from a bodily fluid. In some embodiments, the bodily fluid comprises blood, urine, or saliva. In some embodiments, the plurality of cells comprises skin cells or hair cells. In some embodiments, the plurality of cells comprises plant cells. In some embodiments, the plant cells are derived from a leaf or root.

In some embodiments, the first plurality of cells is proliferated in a bulk growth environment. In some embodiments, the first plurality of cells and the duplicates thereof are stratified by growth rate. In some embodiments, the first plurality of cells are stained with carboxyfluorescein succinimidyl ester (CFSE).

In some embodiments, a portion of the nucleic acid molecules of the plurality of partitioned cells sequenced in (d) comprises a plurality of perturbation barcode sequences associated with a plurality of perturbations. In some embodiments, the plurality of perturbations are selected from the group consisting of the addition of a small molecule, a knockout, an antibody, cell-cell interactions, ribonucleic acid interference (RNAi), an open reading frame (ORF), and Clustered Regularly Interspaced Short Palindromic Repeats (CRISPR) single guide ribonucleic acid (sgRNA). In some embodiments, the plurality of perturbations comprise a variation in temperature and/or a variation in pH. In some embodiments, the plurality of perturbations comprise the introduction of mutated forms of genes.

In some embodiments, a portion of the nucleic acid molecules of the plurality of partitioned cells sequenced in (d) comprises a plurality of barcode sequences associated with a plurality of measurements. In some embodiments, the plurality of measurements are selected from the group consisting of ribonucleic acid sequencing (RNA-seq), Assay for Transposase-Accessible Chromatin using sequencing (ATAC-seq), in-situ sequencing, and cell morphology measurements.

In another aspect, the present disclosure provides a method of analyzing a plurality of cells, comprising: (a) obtaining the plurality of cells from a plurality of subjects; (b) differentially tagging the plurality of cells according to their subject of origin; (c) sequencing nucleic acid molecules of the plurality of cells to provide a plurality of sequencing reads; and (d) assigning common sequencing reads of the plurality of sequencing reads to a given subject of the plurality of subjects, wherein assigning the sequencing reads is done independent of variation among the plurality of cells, wherein the plurality of cells is proliferated in a bulked growth environment.

In some embodiments, differentially tagging the plurality of cells comprises introducing a plurality of barcode sequences to the plurality of cells.

In some embodiments, the plurality of barcode sequences is incorporated into the first plurality of cells via transduction. In some embodiments, the plurality of barcode sequences are incorporated into the first plurality of cells using a viral vector, homologous recombinant integration, Agrobacterium mediated gene transfer, or an episomal vector.

In some embodiments, each barcode sequence of the plurality of barcode sequences comprises between 1 and 1000 bases.

In some embodiments, the plurality of subjects comprises a plurality of human subjects. In some embodiments, the identities of the plurality of subjects are encrypted. In some embodiments, the plurality of cells is derived from a bodily fluid. In some embodiments, the bodily fluid comprises blood, urine, or saliva. In some embodiments, the plurality of cells comprises skin cells or hair cells. In some embodiments, the plurality of cells comprises plant cells. In some embodiments, the plant cells are derived from a leaf or root.

In some embodiments, the plurality of cells is stratified by growth rate. In some embodiments, the plurality of cells are stained with carboxyfluorescein succinimidyl ester (CFSE).

In some embodiments, the plurality of cells sequenced in (c) comprises a plurality of perturbation barcode sequences associated with a plurality of perturbations. In some embodiments, the plurality of perturbations are selected from the group consisting of the addition of a small molecule, a knockout, an antibody, cell-cell interactions, RNAi, an open reading frame (ORF), and Clustered Regularly Interspaced Short Palindromic Repeats (CRISPR) single guide ribonucleic acid (sgRNA). In some embodiments, the plurality of perturbations comprise a variation in temperature and/or a variation in pH. In some embodiments, the plurality of perturbations comprise the introduction of mutated forms of genes.

In some embodiments, the plurality of cells comprise a plurality of barcode sequences associated with a plurality of measurements. In some embodiments, the plurality of measurements are selected from the group consisting of RNA-seq, ATAC-seq, in-situ sequencing, and cell morphology measurements.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an overview of a pooled screening scheme in which cells derived from a plurality of subjects are barcoded en masse (top). Phenotypic profiling may be performed in a pooled format (by association with the barcode) to establish baseline states (bottom left) as well as states in response to perturbations (bottom right). Shading of subject 110 corresponds to shading of cell 111, barcoded cell 112, row 113, and rows 114. Shading of subject 120 corresponds to shading of cell 121, barcoded cell 122, row 123, and rows 124. Shading of subject 130 corresponds to shading of cell 131, barcoded cell 132, row 133, and rows 134.

FIG. 2 schematically illustrates an encryption or ambiguation scheme in which samples and genetic data may be derived from a donor, preserving the donor's access to the results, but maintaining anonymity to those generating the data.

FIG. 3 shows an overview of the methods described herein. Panel A shows an exemplary pooling schema in which cost of deriving cells from large number of donors is reduced, samples can be rejected if contaminated, and stratified by growth rate. Panel B schematically illustrates how deoxyribonucleic acid (DNA)/ribonucleic acid (RNA) barcodes preserve donor identity despite cells from many donors being mixed together. Panel C schematically illustrates how barcodes can be co-associated with DNA sequencing data so that a barcode is uniquely mapped to a genotype. Panel D schematically illustrates a combinatorial co-association approach for mapping perturbations to DNA barcode or many perturbations with one another.

FIG. 4 schematically illustrates a single-cell sequencing scheme.

FIG. 5 schematically illustrates a deconvolution sequencing scheme.

FIG. 6 shows a computer system that is programmed or otherwise configured to implement methods provided herein.

FIG. 7 shows gene expression signatures of cells subjected to a panel of drugs and conditions.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “sample,” as used herein, generally refers to a biological sample. The sample may be of a subject. The sample may include a cell or a plurality of cells. The sample may include a nucleic acid molecule or a plurality of nucleic acid molecules. Nucleic acid molecules may be ribonucleic acid (RNA) or deoxyribonucleic acid (DNA) molecules. The sample may include cells and nucleic acid molecules (e.g., cells containing DNA and RNA). The sample may be a tissue sample. The sample may be a cell-free (or cell free) sample.

The term “subject,” as used herein, generally refers to an individual from whom a sample is obtained. The subject may be a mammal, such as a human, or a plant (e.g., yeast). The subject may be prokaryotic organism (e.g., bacteria) or a eukaryotic organism (e.g., fungus or yeast). The subject may be an animal, such as a farm animal (e.g., goat or pig), dog, cat, mouse, squirrel, or bird. The subject may be symptomatic with respect to a disease (e.g., cancer). The subject may be asymptomatic with respect to the disease. The subject may be patient.

The term “sequencing,” as used herein, generally refers to methods and technologies for determining the sequence of nucleotide bases in one or more nucleic acid molecules (e.g., polynucleotides). The nucleic acid molecules can be, for example, deoxyribonucleic acid (DNA) or ribonucleic acid (RNA), including variants or derivatives thereof (e.g., single stranded DNA). Sequencing may be performed by any available technique. For example, sequencing may be performed by high-throughput sequencing, pyrosequencing, sequencing-by-ligation, sequencing by synthesis, sequencing-by-hybridization, ribonucleic acid sequencing (RNA-Seq) (Illumina), Digital Gene Expression (Helicos), next generation sequencing, single molecule sequencing (e.g., Pacific Biosciences of California and Oxfor Nanopore), single molecule sequencing by synthesis (SMSS) (Helicos), massively-parallel sequencing, clonal single molecule Array (Solexa), shotgun sequencing, Maxim-Gilbert sequencing, primer walking, or Sanger sequencing. Sequencing can be performed by various systems, such as, without limitation, a sequencing system by Illumina, Pacific Biosciences (PacBio), Oxford Nanopore, or Life Technologies (Ion Torrent). Alternatively or in addition to, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a cell or a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced.

Whenever the term “at least,” “greater than,” or “greater than or equal to” precedes the first numerical value in a series of two or more numerical values, the term “at least,” “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “at most”, “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Provided herein are methods of analyzing a plurality of cells. A method may comprise providing a plurality of cells from a plurality of subjects (e.g., humans, plants, or animals), wherein the plurality of cells comprise a plurality of nucleic acid molecules (e.g., deoxyribonucleic acid (DNA) or ribonucleic acid (RNA) molecules). The plurality of cells may be derived from cells of the plurality of subjects. The plurality of nucleic acid molecules may comprise a plurality of barcode sequences. For example, a (e.g., each) nucleic acid molecule of the plurality of nucleic acid molecules may comprise a barcode sequence of the plurality of barcode sequences. In some cases, a barcode sequence of the plurality of barcode sequences may be different from every other barcode sequence. In other cases, the plurality of barcode sequences may comprise multiple copies of the same barcode sequence. The plurality of barcode sequences may be endogenous to the plurality of cells, or may be introduced to the plurality of cells via, for example, transduction or transfection. Nucleic acid molecules of the plurality of nucleic acid molecules of the plurality of cells may then be sequenced (e.g., using next generation sequencing). Nucleic acid molecules derived from the plurality of nucleic acid molecules of the plurality of cells may then be sequenced (e.g., using next generation sequencing). Sequencing may generate a plurality of sequencing reads corresponding to the plurality of nucleic acid molecules. A portion of the plurality of sequencing reads may comprise some or all barcode sequences of the barcode sequences of the plurality of barcode sequences. The plurality of sequencing reads may be processed. The plurality of sequencing reads may comprise the plurality of barcode sequences. The barcode sequence of the plurality of barcode sequences may be used to associate a sequencing read of the plurality of sequencing reads or a subset of the plurality of sequencing reads with a subject of the plurality of subjects from which the plurality of cells derived. In some cases, the plurality of cells may be proliferated in a bulk growth environment. In some cases, the plurality of cells may be generated upon proliferating the cells of the plurality of subjects in a bulk growth environment. In some cases, prior to sequencing, the plurality of nucleic acid molecules may be processed to generate the nucleic acid molecules. The nucleic acid molecules may be subsequently sequenced. The processing may comprise generating copies of the plurality of nucleic acid molecules. The processing may comprise recovering the plurality of nucleic acid molecules from the plurality of cells.

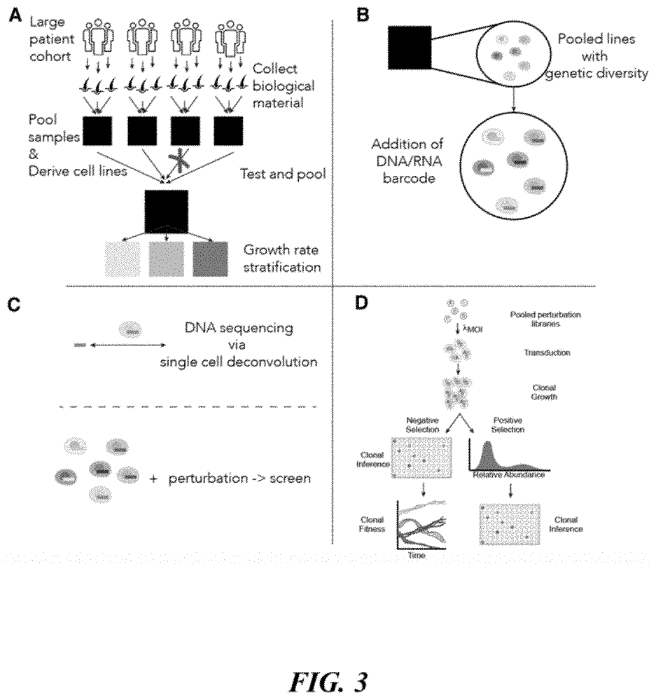

In some cases, a method of analyzing a plurality of cells may comprise providing a first plurality of cells from a plurality of subjects (e.g., humans, plants, or animals), wherein the first plurality of cells comprise a first plurality of nucleic acid molecules (e.g., deoxyribonucleic acid (DNA) or ribonucleic acid (RNA) molecules). The first plurality of cells may be derived from cells of the plurality of subjects. The first plurality of nucleic acid molecules (e.g., a subset of the first plurality of nucleic acid molecules) may comprise a plurality of barcode sequences (e.g., a first plurality of barcode sequences). For example, a nucleic acid molecule of the plurality of nucleic acid molecules may comprise a barcode sequence of the plurality of barcode sequences. In some cases, a barcode sequence of the plurality of barcode sequences may be different from every other barcode sequence. In other cases, the plurality of barcode sequences may comprise multiple copies of the same barcode sequence. The plurality of barcode sequences (e.g., the first plurality of barcode sequences) may be endogenous to the first plurality of cells, or may be introduced to the first plurality of cells via, for example, transduction or transfection. The first plurality of cells may be subjected to conditions sufficient to duplicate cells of the first plurality of cells to provide a second plurality of cells comprising cells of the first plurality of cells and duplicates thereof In some cases, a cell may be duplicated one or more times. The second plurality of cells may comprise a second plurality of nucleic acid molecules comprising some or all barcode sequences of the plurality of barcode sequences (e.g., a second plurality of barcode sequences). Cells of the first plurality of cells and the second plurality of cells may be partitioned between a plurality of partitions (e.g., droplets or wells), thereby providing a plurality of partitioned cells. In some cases, a partition of the plurality of partitions may comprise at most one cell. In other cases, a partition of the plurality of partitions may comprise at least one cell. Nucleic acid molecules of the plurality of partitioned cells may then be sequenced (e.g., using next generation sequencing). Nucleic acid molecules derived from the plurality of partitioned cells may then be sequenced (e.g., using next generation sequencing). Sequencing may generate a plurality of sequencing reads corresponding to the plurality of nucleic acid molecules (e.g., the second plurality of nucleic acid molecules) of the plurality of partitioned cells. A portion of the plurality of sequencing reads may comprise some or all barcode sequences of the barcode sequences of the plurality of barcode sequences (e.g., the plurality of barcode sequences). The plurality of sequencing reads may be processed. The plurality of sequencing reads may comprise the second plurality of barcode sequences. A barcode sequence of the plurality of barcode sequences (e.g., second plurality of barcode sequences) may be used to associate a sequencing read of the plurality of sequencing reads or a subset of the plurality of sequencing reads with a subject of the plurality of subjects from which the first plurality of cells derived. In some cases, prior to sequencing, the plurality of nucleic acid molecules (e.g., the second plurality of nucleic acid molecules) may be processed to generate the nucleic acid molecules. The nucleic acid molecules may be subsequently sequenced. The processing may comprise generating copies of the plurality of nucleic acid molecules (e.g., the second plurality of nucleic acid molecules). The processing may comprise recovering the plurality of nucleic acid molecules (e.g., the second plurality of nucleic acid molecules) from the plurality of cells (e.g., the second plurality of cells). The methods described herein may allow a diverse set of cell clones derived from a plurality of donors to be analyzed at costs and times similar to that required to analyze a sample from a single donor while limiting sample loss due to contamination (see, e.g., panel A of FIG. 3).

### Samples

A plurality of cells for analysis according to the methods provided herein may be derived from a single subject or a plurality of subjects. In some cases, the same number of cells may be derived from a subject of the plurality of subjects. For example, a single cell may be provided for a subject of the plurality of subjects. In other cases, a different number of cells may be derived from a subject of the plurality of subjects. In some cases, cells may be provided in a volume of a material derived from a subject, and the same volume of material may be derived from a subject of the plurality of subjects.

A subject may be any entity having nucleic acid molecules of potential interest. For example, a subject may comprise an organism, such as a unicellular or multicellular organism. A subject may comprise a human, animal, or plant. In an example, a subject may be a human. A subject may be a patient. A plurality of subjects may comprise a patient population. For example, some or all subjects of the plurality of subjects may have or be suspected of having a disease or disorder. Some or all subjects of the plurality of subjects may be known to have previously had a disease (e.g., cancer or another disease or disorder). Alternatively or in addition to, some or all subjects of the plurality of subjects may have or be suspected of having a similar genetic feature, such as a particular genetic mutation. Alternatively or in addition to, some or all subjects of the plurality of subjects may have been or may be suspected of having been exposed to a pathogen such as a virus or bacteria. Alternatively, some or all subjects of the plurality of subjects may be healthy or believed to be healthy. Some or all subjects of the plurality of subjects may share characteristics such as physical characteristics (e.g., height, weight, body mass index, or other physical characteristic), ethnic or racial heritage, place of birth or residence, nationality, disease or remission state, or other characteristics. Subjects need not be selected based on shared characteristics. For example, subjects may be selected at random and/or to sample a random fraction of a population.

Cells derived from a subject may be of any useful type and may be sampled from any useful feature or portion of a subject. Cells may be stem cells, or cells may be reprogrammed to create stem cell lines (e.g., induced pluripotent stem cells (iPS)). Plant cells may be derived from, for example, a leaf or root of a plant. Cells (e.g., cells other than plant cells) may be derived from a bodily fluid of an organism (e.g., human or animal) such as blood (e.g., whole blood, red blood cells, leukocytes or white blood cells, platelets), plasma, serum, sweat, tears, saliva, sputum, urine, mucus, semen, synovial fluid, breast milk, colostrum, amniotic fluid, bile, interstitial or extracellular fluid, bone marrow, or cerebrospinal fluid. Cells may be derived from a tissue sample such as a skin sample or tumor sample obtained from, for example, an organ of a subject. Cells may be obtained from a subject by, for example, accessing the circulatory system (e.g., intravenously or intraarterially), collecting a secreted biological sample (e.g., stool, urine, saliva, sputum, etc.), surgically extracting a tissue (e.g., biopsy), swabbing, pipetting, and breathing. A sample including cells may undergo processing to isolate cells within the sample. For example, a sample comprising one or more cells from a sample may be subjected to centrifugation, selective precipitation, filtration, permeabilization, isolation, and/or other processes.

Cells derived from a subject may comprise one or more nucleic acid molecules. A nucleic acid molecule may comprise a single strand or may be double-stranded. Examples of nucleic acid molecules include, but are not limited to, DNA, genomic DNA, plasmid DNA, complementary DNA (cDNA), cell-free (e.g., non-encapsulated) DNA (cfDNA), cell-free fetal DNA (cffDNA), circulating tumor DNA (ctDNA), nucleosomal DNA, chromatosomal DNA, mitochondrial DNA (miDNA), RNA, messenger RNA (mRNA), transfer RNA (tRNA), micro RNA (miRNA), ribosomal RNA (rRNA), circulating RNA (cRNA), short hairpin RNA (shRNA), small interfering RNA (siRNA), an artificial nucleic acid analog, recombinant nucleic acid, plasmids, viral vectors, and chromatin. Cells derived from a subject may comprise one or more DNA molecules and/or one or more RNA molecules. Nucleic acid molecules of interest may be selected for analysis using, for example, the methods described herein. For example, RNA molecules may be reverse transcribed using a reverse transcription process to generate cDNA, which may be subjected to subsequent analysis.

Nucleic acid molecules may comprise one or more mutations (e.g., somatic or germline mutations). For example, a nucleic acid molecule may include one or more modifications such as one or more additions or deletions. A mutation or modification may be associated with a disease such as a cancer. Examples of mutations include, but are not limited to, additions (e.g., of a single base or base pair or a collection thereof), deletions (e.g., of a single base or base pair or a collection thereof), base substitutions, duplications (e.g., of a single base or base pair or a collection thereof), copy number variations, single nucleotide polymorphisms, gene fusions, transversions, translocations, inversions, indels, DNA lesions, aneuploidy, polyploidy, chromosomal fusions, chromosomal structure alterations, chromosomal lesions, gene amplifications, gene duplications, gene truncations, and base modifications (e.g., methylation).

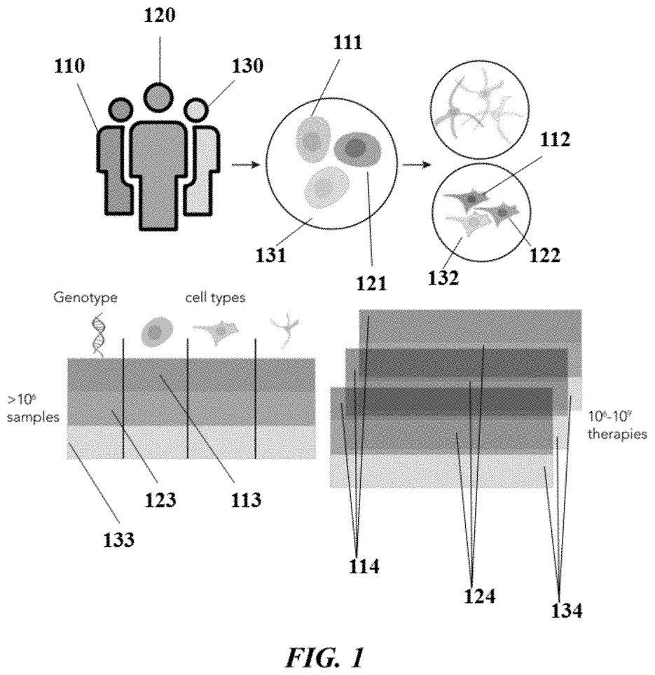

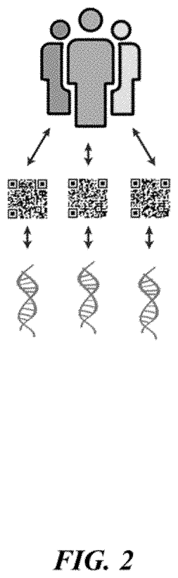

Cells from a plurality of subjects may be pooled into one or more groups (see, e.g., FIG. 1). For example, cells may be pooled into at least about 1, 2, 3, 4, 5, 6, 7, 8, 9, 10, or more groups. The cells may be pooled into less than or equal to about 10, 9, 8, 7, 6, 5, 4, 3, 2, or less groups. By pooling cells from different subjects, cells may be “de-identified” or disassociated from the subjects from which they derive. An identifying feature such as a tag or barcode (e.g., a single barcode sequence or a plurality of barcode sequences) may be provided to cells from a subject prior to pooling so that details of the cells may be associated with the subjects from which they derive. An encryption or ambiguation scheme may be applied to obfuscate the identities of subjects and maintain anonymity while still preserving the ability to analyze cells from a plurality of subjects en masse and provide details of single cells of the subjects (see, e.g., FIG. 2). Such a scheme may be useful in simultaneously protecting patient histories and identities while still generating useful associations between genotypes and phenotypes of a plurality of subjects. Groups into which cells may be pooled may be sized such that the likelihood of a group being contaminated (e.g., deriving from a patient having an infection) is low while still permitting significant cost savings afforded by pooled analysis and reduced needs to test for contamination.

Prior or subsequent to pooling, cells may undergo processing to alter one or more features of the cells or add or remove one or more materials to or from the cells. For example, cells may undergo processing to include a dye or fluorophore to facilitate, for example, visualization of the cells. A dye or fluorophore may be selected from the group consisting of, but not limited to, SYBR green; SYBR blue; 4′,6-diamidino-2-phenylindole (DAPI); propidium iodine; Hoechst; SYBR gold; ethidium bromide; acridine; proflavine; acridine orange; acriflavine; fluorcoumanin; ellipticine; daunomycin; chloroquine; distamycin D; chromomycin; homidium; mithramycin; ruthenium polypyridyls; anthramycin; phenanthridines and acridines; ethidium bromide; propidium iodide; hexidium iodide; dihydroethidium; ethidium homodimer-1 and -2; ethidium monoazide; 9-amino-6-chloro-2-methoxyacridine (ACMA); Hoechst 33258; Hoechst 33342; Hoechst 34580; 7-aminoactinomycin D (7-AAD); actinomycin D; Quinolinium (LDS751); hydroxystilbamidine; SYTOX Blue; SYTOX Green; SYTOX Orange; POPO-1; POPO-3; YOYO-1; YOYO-3; TOTO-1; TOTO-3; JOJO-1; LOLO-1; BOBO-1; BOBO-3; PO-PRO-1; PO-PRO-3; BO-PRO-1; BO-PRO-3; TO-PRO-1; TO-PRO-3; TO-PRO-5; JO-PRO-1; LO-PRO-1; YO-PRO-1; YO-PRO-3; PicoGreen; OliGreen; RiboGreen; SYBR Gold; SYBR Green I; SYBR Green II; SYBR DX; SYTO-40, -41, -42, -43, -44, -45 (blue); SYTO-13, -16, -24, -21, -23, -12, -11, -20, -22, -15, -14, -25 (green); SYTO-81, -80, -82, -83, -84, -85 (orange); SYTO-64, -17, -59, -61, -62, -60, -63 (red); fluorescein; fluorescein isothiocyanate (FITC); tetramethyl rhodamine isothiocyanate (TRITC); rhodamine; tetramethyl rhodamine; Rhodophyta- phycoerythrin (R-phycoerythrin); Cyanine-2 (Cy-2); Cyanine-3 (Cy-3); Cyanine-3.5 (Cy-3.5); Cyanine-5 (Cy-5); Cyanine-5.5 (Cy-5.5); Cyanine-7 (Cy-7); Texas Red; Phar-Red; allophycocyanin (APC); Sybr Green I; Sybr Green II; Sybr Gold; CellTracker Green; ethidium homodimer I; ethidium homodimer II; ethidium homodimer III; ethidium bromide; umbelliferone; eosin; green fluorescent protein; erythrosin; coumarin; methyl coumarin; pyrene; malachite green; stilbene; lucifer yellow; cascade blue; dichlorotriazinylamine fluorescein; dansyl chloride; fluorescent lanthanide complexes such as those including europium and terbium; carboxy tetrachloro fluorescein; 5 and/or 6-carboxy fluorescein (FAM); VIC, 5- (or 6-) iodoacetamidofluorescein; carboxyfluorescein succinimidyl ester (CFSE); 5-((2(and 3)-5-(Acetylmercapto)-succinyl)amino)fluorescein (SAMSA-fluorescein); lissamine rhodamine B sulfonyl chloride; 5 and/or 6 carboxy rhodamine (ROX); 7-amino-methyl-coumarin; 7-Amino-4-methylcoumarin-3-acetic acid (AMCA); boron-dipyrromethene (BODIPY) fluorophores; 8-methoxypyrene-1,3,6-trisulfonic acid trisodium salt; 3,6-Disulfonate-4-amino-naphthalimide; phycobiliproteins; AlexaFluor 350, 405, 430, 488, 532, 546, 555, 568, 594, 610, 633, 635, 647, 660, 680, 700, 750, and 790 dyes; DyLight 350, 405, 488, 550, 594, 633, 650, 680, 755, and 800 dyes, other fluorophores; Black Hole (BH) Dyes and/or Black Hole Quencher (BHQ) Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, other quenchers (such as Dabcyl and Dabsyl, Cy5Q and Cy7Q and Dark Cyanine dyes (GE Healthcare), Dy-Quenchers (Dyomics) (such as DYQ-660 and DYQ-661), and ATTO fluorescent quenchers (ATTO-TEC GmbH) (such as ATTO 540Q, 580Q, 612Q)). For example, cells may be stained with CFSE. Staining cells with a fluorophore or dye may facilitate identification of different generations of cells (e.g., stratification by growth rate) within a clonal population. Staining may thus reduce bias due to clonal dynamics.

In another example, a plurality of fluorescent probes may be introduced to a plurality of cells (e.g., before or after pooling of cells from different subjects or sample collection conditions or pre-processing conditions). The plurality of cells may be subjected to conditions sufficient to hybridize the plurality of fluorescent probes to a plurality of nucleic acid molecules included in the cells, such as to a plurality of barcode sequences included within the plurality of cells. The plurality of fluorescent probes hybridized to the plurality of nucleic acid molecules (e.g., to the plurality of barcode sequences) may be optically detected (e.g., via imaging). This process may be repeated one or more times with the same or different fluorescent probes (e.g., probes having different nucleic acid sequences and/or different fluorescent moieties). This process may be used to be identify cells via their barcode sequences, and may be particularly useful for barcode sequences comprising two or more barcode segments. This process may comprise fluorescence in situ hybridization (e.g., fluorescence in situ hybridization (FISH), such as sequential fluorescence in situ hybridization (seqFISH)). In some cases, barcode sequences interrogated in such a manner may be of a first set of barcode sequences of a plurality of barcode sequences (e.g., a plurality of barcode sequences endogenous to the plurality of cells or introduced to the plurality of cells, as described herein), and barcode sequences processed using nucleic acid sequencing (e.g., as described herein) may be of a second set of barcode sequences of the plurality of barcode sequences. The first and second sets of barcode sequences may overlap or may be distinct from one another.

Cells may be barcoded prior or subsequent to pooling of cells from a plurality of subjects in order to differentiate between cells from different subjects. This barcoding scheme may facilitate associations between genotype and phenotype at greatly reduced costs relative to single-donor analyses (see, e.g., panel B of FIG. 3). A barcode delivered to a cell prior to subsequent analysis or a barcode that comprises a subset of endogenous variation may be referred to as a “genotype barcode.” For example, a barcode may comprise overlapping modifications and variants such as, for example, single nucleotide polymorphisms (SNPs), indels, and copy number variations. A barcode may comprise a nucleic acid sequence. Such a sequence may comprise any useful number of canonical nucleotides (e.g., nucleotides comprising adenine, cytosine, guanine, thymine, or uracil nucleobases) or non-canonical nucleotides (e.g., nucleotide analogs comprising non-canonical nucleobase, sugar, or linker moieties). For example, a nucleic acid barcode sequence may comprise at least about 2, 3, 4, 5, 6, 7, 8, 9, 10, 11, 12, 13, 14, 15, 16, 17, 18, 19, 20, or more nucleotides or base pairs. A nucleic acid barcode sequence may comprise less than or equal to about 20, 19, 18, 17, 16, 15, 14, 13, 12, 11, 10, 9, 8, 7, 6, 5, 4, 3, 2, or less nucleotides or base pairs. A nucleic acid barcode sequence may comprise, for example, between 6-10 nucleotides or base pairs. A nucleic acid barcode sequence may comprise at least about 10, 50, 100, 1,000, or more nucleotides or base pairs. A nucleic acid barcode sequence may comprise less than or equal to about 1000, 100, 50, 10, or less nucleotides or base pairs. A nucleic acid barcode sequence may comprise from 1 nucleotides or base pairs to 1000 nucleotides or base pairs, such as from 4 to 10, 4 to 20, 4 to 50, 4 to 100, 10 to 100, 10 to 1,000, or 100 to 1,000 nucleotides or base pairs. A barcode may comprise one or more different barcode sequences that may be provided to a cell or nucleic acid molecule at the same or different times. For example, a barcode may comprise a first barcode sequence corresponding to a first parameter (e.g., a row or column position in a well) and a second barcode sequence corresponding to a second parameter. A barcode sequence may comprise two or more barcode segments, such as two or more barcode segments that may be the same or different. Such a barcode sequence may be constructed using a combinatorial assembly method, such as a split pool method. A barcode sequence may be a subset of the endogenous nucleic acids present in the cell. A barcode may be, for example, a DNA barcode or an RNA barcode. A DNA barcode may be expressed as an RNA barcode. A barcode may be provided to a cell using, for example, transfection or transduction. A barcode may be provided to a cell using, for example, an antibody (e.g., an antibody conjugated to the barcode, such as an antibody-conjugated oligonucleotide), Agrobacterium mediated gene transfer, homologous recombination (HR) integration, an episomal vector, or a viral vector. For example, a barcode may be provided to a cell using a virus (e.g., lentivirus, retrovirus, or adenovirus). A large number of barcodes may be provided to the plurality of cells from the plurality of subjects (e.g., greater than 10-fold larger than the number of cells to be barcoded) such that the likelihood of cells derived from different subjects having the same barcode is low. A subject may have a barcode sequence that is different from other subjects (e.g., a subject may have a unique barcode sequence). In some cases, a plurality of cells from a first subject may be barcoded at a first time, under a first set of conditions, and/or using a first set of barcode sequences, while a plurality of cells from a second subject may be barcoded at a second time, under a second set of conditions, and/or using a second set of barcode sequences, which second time, second set of conditions, and/or second set of barcode sequences may be different from the first time, first set of conditions, and/or first set of barcode sequences. In some cases, a first set of barcode sequences may be introduced to cells from different subjects prior to pooling the cells, and then a second set of barcode sequences may be introduced to the cells subsequent to pooling the cells. The barcode sequences of the first set of barcode sequences introduced to cells from a same subject may have the same sequences, while barcode sequences of the second set of barcode sequences introduced to cells from a same subject (e.g., in a pool comprising cells from one or more other subjects) may have different sequences. A barcode may be provided to a cell along with one or more other components. For example, reprogramming factors to create stem cell lines (e.g., induced pluripotent stem cells (iPSs)) may be provided with a barcode (e.g., in the same transfection process, or as components of a barcode).

The present disclosure provides methods for proliferating (e.g., duplicating cells or increasing the number of cells) cells, which may include barcoded nucleic acid molecules (e.g., DNA and/or RNA). Such methods may include subjecting cells to one or more cycles of cell division (e.g., cloning). Such methods may include subjecting cells to cell growth (e.g., replication of genetic materials).

Barcoded cells may be subjected to conditions sufficient for duplication. Duplicates of barcoded cells may comprise the same barcode as the parent cells, thereby enriching the sample population for further analysis. Barcoded cells may be subjected to duplication conditions prior to pooling of cells from different subjects. Alternatively (e.g., where cells have been pooled prior to barcoding), barcoded cells may be subjected to duplication conditions subsequent to pooling of cells from different subjects. Barcoded cells may be cultured in an incubator, on a plate (e.g., microwell plate), in a bioreactor, in a droplet, or in any other vessel or compartment. Temperature, gas mixture, pH, plating density, growth media, and/or other conditions may be selected to optimize growth of a cell type. Staining the cells with a dye such as CFSE may facilitate stratification of cells by growth rate. Cells may then be selected from specific generations (e.g., originally extracted cells, first generation, second generation, third generation, etc.) for further analysis, thereby reducing bias due to clonal dynamics. Cells and duplicates thereof may be pooled. Pooled samples including cells and duplicates thereof may comprise at least about 1, 2, 3, 4, 5, 6, 7, 8, 9, 10, 20, 30, 40, 50, 100, or more copies of an original cell derived from a subject of a plurality of subjects. Pooled samples including cells and duplicates thereof may comprise less than or equal to about 100, 50, 40, 30, 20, 10, 9, 8, 7, 6, 5, 4, 3, 2, or less copies of an original cell derived from a subject of a plurality of subjects. In some cases, a pooled sample may comprise from 1 copy of an original cell to 10,000 copies of an original cell, such as from 1 to 10, from 1 to 100, from 1 to 1,000, from 1 to 5,000, from 10 to 100, from 10 to 1,000, from 10 to 10,000, from 100 to 1,000, from 100 to 10,000, or from 1,000 to 10,000 copies of an original cell. A pooled sample including cells and duplicates thereof may be sampled so that several members of an original cell are sampled. For example, from 1 copy of an original cell to 1,000 copies of an original cell may be sampled. In some cases, all of a pooled sample may undergo the subsequent analysis. In other cases, a portion of a pooled sample may undergo a first analysis and another portion of the pooled sample may undergo a second analysis. For example, a first portion of a pooled sample may undergo nucleic acid sequencing while a second portion of a pooled sample may be interrogated using microscopy or subjected to one or more assays or screens. For example, cells (e.g., of a pooled sample) may undergo drug screening, gene expression screening (e.g., using fluorescence-activated cell sorting (FACS)), or other screening such that the abundance of barcodes associated with a phenotype may be used to associate genotype to phenotype at a large scale. Similarly, screening to identify associations between a barcoded genotype and a single cell phenotype may be performed at scale using, for example, microscopy or single cell sequencing.

In a first example, a plurality of cells may be obtained from a plurality of subjects. A plurality of unique barcodes may be provided to cells from a subject such that a cell from a subject is provided with the same barcode and the cells from different subject are provided with different barcodes. Barcodes (e.g., nucleic acid barcode sequences) may be provided to cells using, for example, a viral vector, such as a lentiviral vector. Barcoded cells may then be subjected to conditions sufficient to duplicate the barcoded cells, and a dye may be used to stratify cells by growth rate (as described elsewhere herein). Alternatively, transient expression of a fluorescent protein may be used to stratify cells by growth rate. Examples of transient expression include, but are not limited to, transient transfection and transiently induced expression through a dox-inducible or cumate-inducible promoter system. Barcoded cells and duplicates thereof from different subjects of the plurality of subjects are then pooled for subsequent analysis.

In a second example, a plurality of cells may be obtained from a plurality of subjects. The cells derived from a subject of the plurality of subjects may then be pooled. A plurality of unique barcodes may be provided to the pooled cells. The number of unique barcodes may be such that a cell should be provided with a different barcode. Barcodes (e.g., nucleic acid barcode sequences) may be provided to cells using, for example, a viral vector such as a lentiviral vector. The pooled barcoded cells may then be subjected to conditions sufficient to duplicate the barcoded cells, and a dye may be used to stratify cells by growth rate. Barcoded cells and duplicates thereof from may then undergo subsequent analysis.

In a third example, a plurality of cells may be obtained from a plurality of subjects. A plurality of unique barcodes may be provided to cells from a subject such that a cell from a subject is provided with the same barcode and the cells from different subject are provided with different barcodes. Barcodes (e.g., nucleic acid barcode sequences) may be provided to cells using, for example, a viral vector such as a lentiviral vector. Barcoded cells may then be pooled. The pooled barcoded cells may then be subjected to conditions sufficient to duplicate the barcoded cells, and a dye may be used to stratify cells by growth rate. Barcoded cells and duplicates thereof from may then undergo subsequent analysis.

### Single Cell Analysis

Barcoded cells may undergo sequencing to analyze nucleic acid molecules included therein. Sequencing a plurality of pooled cells may be computationally and experimentally expensive. Accordingly, the present disclosure provides methods for obtaining sequencing information at a single cellular level at a substantially reduced computational and experimental cost.

Barcoded cells (e.g., from a pooled sample comprising barcoded cells and duplicates thereof derived from a plurality of subjects) may be partitioned between a plurality of partitions. In some cases, the plurality of partitions may comprise a plurality of wells. In other cases, the plurality of partitions may comprise a plurality of droplets (e.g., aqueous droplets). The plurality of partitions may comprise, for example, at least about 2 partitions, such as at least about 10, 20, 30, 40, 50, 60, 70, 80, 90, 100, 1,000, 10,000, 100,000, 1,000,000, 10,000,000, 100,000,000, 1,000,000,000 or more partitions. The plurality of partitions may comprise, for example, less than or equal to about 1,000,000,000 partitions, such as less than or equal to about 100,000,000, 10,000,000, 1,000,000, 100,000, 10,000, 1,000, 100, 90, 80, 70, 60, 50, 40, 30, 20, 10, 5, or less partitions. In some cases, the plurality of partitions may comprise 96 partitions (e.g., 96 wells) or a multiple of 96 partitions (e.g., multiple 96 well plates). In some cases, the plurality of partitions may comprise at least about 1,000 partitions, such as at least about 1,000 aqueous emulsion droplets. Partitions may comprise one or more cells. For example, a partition of a plurality of partitions may comprise a single cell. Alternatively, a partition of a plurality of partitions may comprise more than one cell. In some cases, a partition may not include a cell. For example, a droplet of a plurality of droplets may not comprise a cell. In some cases, a droplet of a plurality of droplets may comprise at most one cell (e.g., 0 or 1 cell). In some cases, a droplet of a plurality of droplets may comprise a fraction of a cell (e.g., between 0 and 1 cell). In other cases, a droplet of a plurality of droplets may comprise one or more cells. In another example, a well of a plurality of wells may not comprise a cell. In some cases, a well of a plurality of wells may comprise at least about 2, 3, 4, 5, 6, 7, 8, 9, 10, or more cells. A well of a plurality of wells may comprise less than or equal to about 10, 9, 8, 7, 6, 5, 4, 3, 2, or less cells.

Cells distributed amongst a plurality of partitions may be co-partitioned with one or more reagents. For example, cells may be co-partitioned with one or more reagents selected from the group consisting of permeabilizing agents, lysis agents or buffers, enzymes (e.g., polymerases, reverse transcriptases, or other enzymes), fluorophores, fluorescent probes, labeling moieties, primer molecules, adapters, barcodes (e.g., nucleic acid barcode molecules), oligonucleotides, buffers, deoxynucleotide triphosphates, reducing agents, oxidizing agents, chelating agents, detergents, stabilizing agents, nanoparticles, beads, and antibodies. In some cases, cells may be transferred to a partition that already includes one or more reagents. In some cases, cells may be transferred to a partition and one or more reagents may subsequently be provided to the partition. In other cases, cells and reagents may be provided to a partition at the same time (e.g., during droplet formation). Partitioned cells may undergo processing including permeabilization and/or lysis to provide access to nucleic acid molecules included therein. For example, cells included within a partition may be brought into contact with a lysis agent to release nucleic acid molecules from the cells and make them available for further processing. Alternatively, cells may be permeabilized to provide access to nucleic acid molecules therein. In some cases, RNA molecules may undergo reverse transcription. For example, RNA molecules may be brought into contact with a reverse transcriptase to provide cDNA molecules. In some cases, nucleic acid molecules included within partitions may be duplicated by, for example, a nucleic acid extension or amplification reaction. A primer molecule may hybridize to a nucleic acid molecule and the resultant complex may undergo a primer extension reaction. A polymerase (e.g., a DNA or RNA polymerase) and nucleotides (e.g., deoxyribonucleotide triphosphate (dNTPs)) may be used in the primer extension reaction. Alternatively, a primer molecule or adapter may be ligated to an end of a nucleic acid molecule and be used as a basis for an amplification reaction. Any useful nucleic acid amplification reaction may be used. In some cases, polymerase chain reaction (PCR) (e.g., digital PCR, real time PCR, or quantitative PCR) may be used to amplify nucleic acid molecules included within a partition. In some cases, an isothermal amplification reaction may be used to amplify nucleic acid molecules included within a partition.

Primer molecules and adapters used in nucleic acid duplication reactions may comprise random Nmer sequences. The use of such sequences may facilitate amplification of potentially unknown sequences of nucleic acid molecules included within partitions. Alternatively, or in addition, primer molecules and adapters may comprise targeted Nmer sequences (e.g., poly(T) sequences). In some cases, both random and targeted Nmer sequences may be used. Primer molecules and adapters may be of any useful length and have any useful features. For example, a primer molecule or adapter may comprise a fluorophore or other labelling moiety that may be optically detected or otherwise used to identify the sequence to which the primer molecule or adapter attaches. In some cases, a primer molecule or adapter may comprise a barcode sequence (e.g., as described herein) or unique molecular identifier (UMI) sequence. Such a sequence may alternatively be referred to herein as a “cellular barcode.” A primer molecule or adapter may also comprise one or more additional sequences including one or more sequencing primers (e.g., sequences useful for sequencing platform, such as Illumina P5 and P7 sequences) or other functional sequences to facilitate analysis of nucleic acid molecules by, for example, sequencing.

Nucleic acid molecules may undergo single-cell sequencing (e.g., RNA sequencing, RNA-seq) and/or other processing such as other single cell assays. For example, nucleic acid molecules may also be analyzed using Assay for Transposase-Accessible Chromatin using sequencing (ATAC-seq).

### Single-Cell Sequencing

In some cases, partitioned cells may be subjected to single-cell sequencing. Partitioned cells may be provided a cellular barcode that is unique to a cell. In some cases, the number of cells associated with a cellular barcode may be greater than one such that at least about 2, 3, 4, 5, 6, 7, 8, 9, 10, or more cells may be associated with a cellular barcode. In some cases, the number of cells associated with a cellular barcode may be less than 20 such that less than or equal to about 10, 9, 8, 7, 6, 5, 4, 3, 2, or less cells may be associated with a cellular barcode. Sequencing may be performed to associate sequences of nucleic acid molecules of partitioned cells (e.g., genomic DNA sequences) with cellular barcodes. In an example, cells may be partitioned amongst a plurality of partitions (e.g., droplets) such that a partition includes no more than one cell. Cells may be co-partitioned with reagents useful for barcoding and/or further processing a cell. For example, cells may be co-partitioned with a bead comprising a plurality of nucleic acid barcode molecules attached thereto. A nucleic acid barcode molecule may comprise a priming sequence as well as a barcode sequence that is unique to that bead and that is the same across all nucleic acid barcode molecules of the plurality of nucleic acid barcode molecules attached to the bead. In this manner, a different cell within a different partition may be provided a unique cellular barcode. The cellular barcode may be provided to the cell via, for example, transduction or transfection (e.g., as described elsewhere herein) or as a component of a primer molecule or adapter that hybridizes or ligates to a nucleic acid molecule of the cell. In the latter case, the nucleic acid barcode molecules attached to the bead may be released from the bead (e.g., by application of a stimulus, such as a photo, thermal, or chemical stimulus) to facilitate interaction between the nucleic acid barcode molecules and nucleic acid molecules of the cell. The use of random priming sequences (e.g., random Nmers) may allow a wide range of sequences of nucleic acid molecules to be sampled. All or portions of nucleic acid molecules (e.g., nucleic acid molecules with primers or adapters hybridized or ligated thereto) may be duplicated within their respective partitions (e.g., via a primer extension reaction). Following interaction of nucleic acid molecules of a cell of a partition with nucleic acid barcode molecules co-partitioned with the cell (e.g., attached to a bead), the partition may comprise a plurality of barcoded nucleic acid sequences. A barcoded nucleic acid sequence may comprise a sequence of a nucleic acid molecule of the partitioned cell, or a complement thereof; the cellular barcode, or a complement thereof; and, in some cases, one or more sequencing primers. Some, but not all, barcoded nucleic acid sequences of a partition may comprise the genotype barcode. In some cases, a barcoded nucleic acid sequence may comprise a first sequencing primer at a first end and a second sequencing primer at a second end. The sequence of the nucleic acid molecule of the partitioned cell and the cellular barcode sequence, or complements thereof, may be disposed between the first and second sequencing primers. Barcoded nucleic acid sequences of different partitions of a plurality of partitions may be pooled (e.g., by combining droplets) and provided to a sequencer (e.g., an Illumina sequencer). In some cases, sequencing primers and/or other functional sequences may be provided to barcoded nucleic acid sequences subsequent to release of the barcoded nucleic acid sequences from their respective partitions, after which the further processed barcoded nucleic acid sequences may undergo sequencing.

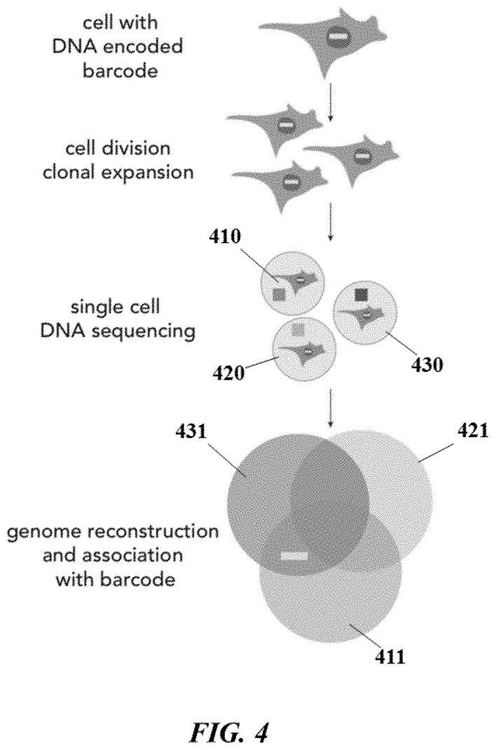

Barcoded nucleic acid sequences may be sequenced to generate a plurality of sequencing reads (e.g., FIG. 4). The plurality of sequencing reads may then be processed to associate genomic DNA sequences with cellular barcodes. A reconstruction approach may be applied such that partial or incomplete genomes from a cell may be combined into a complete or more complete genome sequence of the original cell associated with a genotype barcode (see, e.g., FIG. 4). In FIG. 4, shading of 410 corresponds to shading of 411, shading of 420 corresponds to shading of 421, and shading of 430 corresponds to shading of 431. The reconstruction approach may identify overlap between genotype barcodes and cellular barcodes and use this information to determine that some or all sequencing reads including a cellular barcode originated from a shared ancestor cell. Overlapping modifications and variants such as, for example, single nucleotide polymorphisms (SNPs), indels, and copy number variations associated with different cellular barcodes may also be used to determine that some or all of the sequencing reads having such features originated from a shared ancestor cell. Notably, the overlapping modification and variants may themselves be used as endogenous “genotype barcodes.” For example, a first cell may have associated therewith a first genotype barcode and a first cellular barcode, while a second cell that is a duplicate of the first cell may have associated therewith the same first genotype barcode and a second cellular barcode that is different from the first cellular barcode. By determining the genotype barcode associated with the first and second cellular barcodes, the first and second cells may be determined to be of the same origin. If the genotype barcode has been associated with a subject, the first and second cells may further be attributed to the subject. In another example, a first sequencing read including a first cellular barcode and a second sequencing read including a second cellular barcode that is different from the first cellular barcode may include the same SNP. The overlapping SNP may be used to determine that the two sequencing reads are associated with the same ancestor cell and thus with the same subject. In some cases, a reconstruction approach may use or establish a threshold to determine whether a significant amount of overlap in DNA variants exists. For example, the reconstruction approach may use a threshold at which a significant amount of overlap in DNA variants is determined based on the likelihood that two identical genotype barcodes are correctly paired. In some cases, genotype barcodes may be corrected for one or more modifications (e.g., one or more mutations, such as at least about 1, 2, 3, 4, 5, 6, 7, 8, 9, 10, or more mutations), e.g., using a reconstruction approach described above. In some cases, genotype barcodes may be corrected for modifications (e.g., mutations, such as less than or equal to about 10, 9, 8, 7, 6, 5, 4, 3, 2, or less mutations), e.g., using a reconstruction approach described above. Similarly, in some cases, cellular barcodes may be corrected for one or more modifications (e.g., one or more mutations, such as 1, 2, 3, 4, 5, 6, 7, 8, 9, 10, or more mutations), e.g., using a reconstruction approach described above. The cellular barcodes may be corrected for modifications (e.g., mutations, such as less than or equal to about 10, 9, 8, 7, 6, 5, 4, 3, 2, or less mutations), e.g., using a reconstruction approach described above. Further, the single-cell sequencing method may be used to simultaneously process a plurality of cells, such as, for example, at least about 2, 5, 10, 50, 100, 1,000, or more cells. The single-cell sequencing method may be used to simultaneously process a plurality of cells, such as, for example, less than or equal to about 1000, 100, 50, 10, 5, 2, or less cells. For example, from 2 cells to 10 cells, 10 cells to 100 cells, or 100 cells to 1,000 cells may be simultaneously processed. Accordingly, the method provided herein facilitates single-cell sequencing on a massive scale.

In some cases, an external dataset may be used to facilitate reconstruction. For example, if only 100 single nucleotide polymorphisms (SNPs) are observed in a sample, the amount of overlap between two samples may be close to 0. However, when compared to an external database of SNPs such as the Exome Aggregation Consortium (ExAC) or 1,000 genomes, reconstructions may still be possible.

In some cases, information regarding genomic DNA sequences may be ascertained using DNA variants detected during RNA sequencing. The frequency of variants for regions of DNA (genomic or otherwise) may serve as a barcode or a component of a barcode. For example, the frequency of alleles in mitochondrial DNA and/or the insertion of multiple exogenous barcodes may serve as a barcode or a component of a barcode. Sequencing involving deconvolution

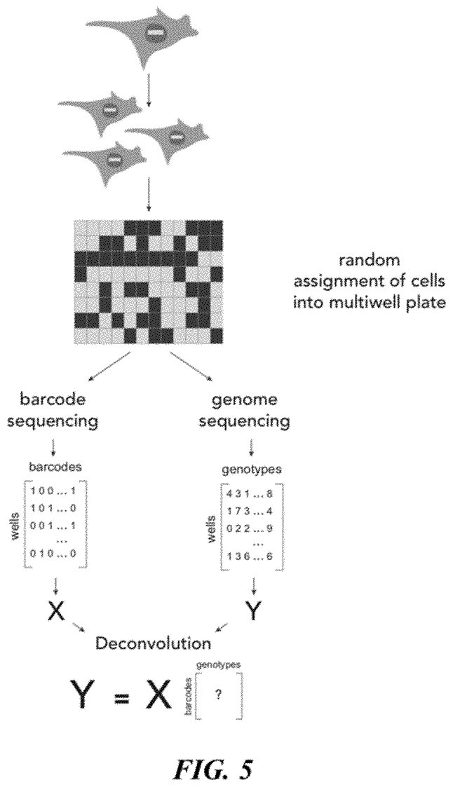

In some cases, partitioned cells may undergo a multiplexed sequencing method comprising a deconvolution process (see, e.g., FIG. 5). Cells may be partitioned between a plurality of partitions (e.g., 10 or more partitions, such as at least about 10, 20, 100, 1,000, 10,000, 100,000, or more partitions) such that a partition of the plurality of partitions comprises one or more cell. Cells may be partitioned between a plurality of partitions (e.g., such as less than or equal to about 100,000, 10,000, 1,000, 100, 20, 10, or less partitions) such that a partition of the plurality of partitions comprises one or more cell. The probability that cells corresponding to different original (e.g., ancestral) cells may be present in the same combination of partitions may be low. For example, there may be less than a 1 in 10,000,000,000 chance that cells present in 7 wells out of a 96 well plate will be present in the same set of wells. The cells included within a partition (e.g., well) may be permitted to divide within the partition to provide more material for subsequent analysis. Cells may be lysed or permeabilized within their respective partitions to provide access to nucleic acid molecules therein. The resultant partition contents (e.g., lysate) may then be processed for sequencing such that a partition may be labeled with a unique partition barcode. A partition barcode may be provided in the same manner as the genotype barcode (e.g., as described elsewhere herein) if cells are not lysed. Alternatively, a partition barcode may be provided via, for example, a nucleic acid barcode molecule that may comprise a partition barcode as well as, in some cases, additional sequences. Such nucleic acid barcode molecules may be provided in solution or attached to a substrate such as a bead. In some cases, nucleic acid barcode molecules comprising partition barcode sequences may be included within partitions prior to addition of cells (e.g., within solution or immobilized to a surface of a partition, such as a portion of a well of a multiwell plate). In some cases, a nucleic acid barcode molecule may include a partition barcode as well as a priming sequence (e.g., a targeted or random priming sequence, as described elsewhere herein). The priming sequence of the nucleic acid barcode molecule may hybridize or ligate to nucleic acid molecules included within a partition. Nucleic acid molecules included within a partition (e.g., nucleic acid molecules hybridized or ligated to nucleic acid barcode molecules) may undergo one or more duplication processes such as one or more primer extension reactions or nucleic acid amplification reactions. Following interaction of nucleic acid molecules of a partition with nucleic acid barcode molecules provided to the partition, the partition may comprise a plurality of barcoded nucleic acid sequences. A barcoded nucleic acid sequence may comprise a sequence of a nucleic acid molecule of one of the cells partitioned within the partition, or a complement thereof; the partition barcode, or a complement thereof; and, in some cases, one or more sequencing primers. Some, but not all, barcoded nucleic acid sequences of a partition may comprise a genotype barcode. In some cases, a barcoded nucleic acid sequence may comprise a first sequencing primer at a first end and a second sequencing primer at a second end. The sequence of a nucleic acid molecule of a partitioned cell and the partition barcode sequence, or complements thereof, may be disposed between the first and second sequencing primers. Barcoded nucleic acid sequences of different partitions of a plurality of partitions may be pooled and provided to a sequencer (e.g., an Illumina sequencer). In some cases, sequencing primers and/or other functional sequences may be provided to barcoded nucleic acid sequences subsequent to release of the barcoded nucleic acid sequences from their respective partitions, after which the further processed barcoded nucleic acid sequences may undergo sequencing.

Barcoded nucleic acid sequences may be sequenced to generate a plurality of sequencing reads. The plurality of sequencing reads may then be processed to associate genomic DNA sequences from a partition (e.g., well) with its corresponding partition barcode. In some cases, long read sequencing may be employed to facilitate more accurate reconstruction of genomic information. The frequency of modifications and variants such as, for example, single nucleotide polymorphisms (SNPs), indels, and copy number variations of sequencing reads associated with a partition may also be determined. A reconstruction approach may be applied in which sequences associated with a genotype barcode may be determined in a manner that maximizes the observed frequencies of DNA variants across partitions of the plurality of partitions. The reconstruction approach may comprise the use of maximum likelihood, multivariate regression, clustering, and/or neural networks. Any prior information about genetic covariation may be used to improve reconstruction accuracy. The accuracy of a reconstruction approach may be improved to using long read sequencing to more accurately determine the co-occurrence of modifications and variants. In some cases, a reconstruction approach involving short read sequencing may use barcodes to phase. The reconstruction approach may provide for determination of associations between genotype barcodes and partition barcodes and may thus facilitate construction of complete or partially complete genome sequences of the original cells associated with genotype barcodes. For example, a first sequencing read deriving from a first cell of a first partition may have associated therewith a first genotype barcode and a first partition barcode, while a second sequencing read deriving from a second cell of a second partition may have associated therewith the same first genotype barcode (e.g., the second cell may be a duplicate of the first cell, or vice versa) and a second partition barcode that is different from the first partition barcode. Both, one, or neither sequencing read may include its respective genotype barcode. A reconstruction technique may be employed to identify a feature of the first sequencing read of the first partition and a feature of the second sequencing read of the second partition as being the same, and to then identify the first and second sequencing reads as being associated with the same ancestral cell. In some cases, genotype barcodes may be corrected for one or more modifications (e.g., one or more mutations, such as at least about 1, 2, 3, 4, 5, 6, 7, 8, 9, 10, or more mutations), e.g., using a reconstruction approach described above. In some cases, genotype barcodes may be corrected for modifications (e.g., mutations, such as less than or equal to about 10, 9, 8, 7, 6, 5, 4, 3, 2, or less mutations), e.g., using a reconstruction approach described above. Similarly, in some cases, partition barcodes may be corrected for one or more modifications (e.g., one or more mutations, such as at least about 1, 2, 3, 4, 5, 6, 7, 8, 9, 10, or more mutations), e.g., using a reconstruction approach described above. The partition barcodes may be corrected for modifications (e.g., mutations, such as less than or equal to about 10, 9, 8, 7, 6, 5, 4, 3, 2, or less mutations), e.g., using a reconstruction approach described above. Further, the deconvolution-based sequencing method may be used to simultaneously process a plurality of cells, such as, for example, at least about 2, 5, 10, 50, 100, 1,000, or more cells. The deconvolution-based sequencing method may be used to simultaneously process a plurality of cells, such as, for example, less than or equal to about 1000, 100, 50, 10, 5, 2, or less cells. For example, from 2 cells to 10 cells, 10 cells to 100 cells, or 100 cells to 1,000 cells may be simultaneously processed. Accordingly, the method provided herein facilitates single-cell sequencing on a massive scale.

### Perturbations

In some cases, a perturbation may be coupled to a genotype across a plurality of cells (see, e.g., panel C of FIG. 3). For example, a genetic, drug, or environmental perturbation may be coupled to a barcode (e.g., a DNA barcode that is may be expressed as an RNA barcode) and integrated into the genome of cells of a plurality of cells as described in the preceding sections. A perturbation may comprise, for example, the addition of a small molecule, a knockout, open reading frame (ORF), or Clustered Regularly Interspaced Short Palindromic Repeats (CRISPR) single guide RNA (sgRNA). In some cases, a perturbation may comprise a variation in temperature or pH. By associating a genotype barcode (e.g., a barcode associated with a subject) with a perturbation barcode, an association between genotype and perturbation may be determined. This association may be used to identify a cellular response, such as transcriptomic changes (through RNA sequencing) and/or morphology (if sequencing is performed in situ).

A perturbation barcode may be a nucleic acid barcode. In some cases, a perturbation barcode may comprise a nucleic acid sequence that identifies another transduced element, such as an open reading frame (ORF), guide RNA (e.g., sgRNA), or short hairpin RNA. In some cases, the perturbation barcode may be provided to the cell using, for example, transfection or transduction. In some cases, a perturbation barcode may be provided to a cell using an antibody (e.g., an antibody conjugated to the barcode, such as an antibody-conjugated oligonucleotide), Agrobacterium mediated gene transfer, homologous recombination (HR) integration, an episomal vector, or a viral vector. For example, a perturbation barcode may be provided to a cell using a virus (e.g., lentivirus, retrovirus, or adenovirus). In some cases, a perturbation barcode may be used in addition to a genotype barcode. Single-cell sequencing (e.g., as described above) may be used to associate a genotype barcode with both one or more perturbation barcodes and a cellular barcode to establish an association between genotype and perturbations. Alternatively, a deconvolution approach may be used in which clonal expansion may be followed by random assortment of cells between a plurality of partitions (e.g., across a multiwell plate) and correlations between barcodes derived using a deconvolution/reconstruction approach. Sequencing of one or more perturbation barcodes may be performed in such a way that associates it with a partition barcode. A genotype barcode may also be sequenced so that it may be associated with a partition barcode to establish an association between genotype and perturbation. Details of single-cell sequencing and deconvolution approaches are included elsewhere herein.

### Computer Systems

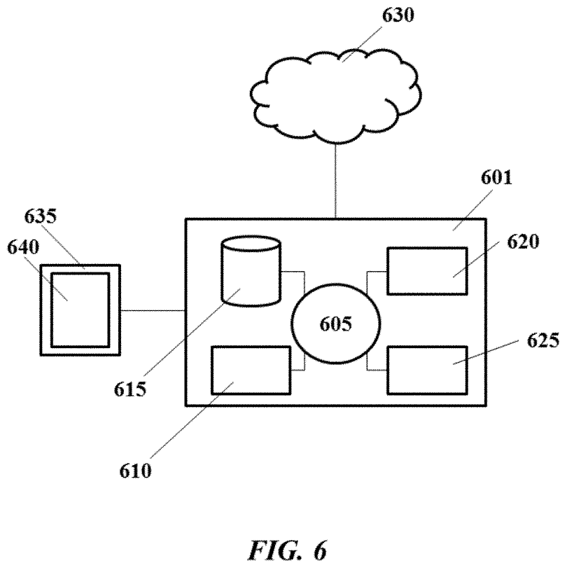

The present disclosure provides computer systems that are programmed to implement methods of the disclosure. FIG. 6 shows a computer system 601 that is programmed or otherwise configured to carry out the methods provided herein. The computer system 601 can regulate various aspects of the methods of the present disclosure, such as, for example, pooling of cells from different samples, partitioning of cells between a plurality of partitions, providing barcodes to cells within or outside of partitions, sequencing of sequencing reads, and determining associations between genotypes and phenotypes. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (PC) (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a compact disc read-only memory (CD-ROM), digital versatile disc (DVD) or digital versatile disk - read only memory (DVD-ROM), any other optical medium, punch cards paper tape, any other physical storage medium with patterns of holes, a random access memory (RAM), a read-only memory (ROM), a programmable read-only memory (PROM) and erasable programmable read-only memory (EPROM), a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, visualizations of barcodes and variants amongst a plurality of partitions and/or associations between genotypes and phenotypes. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, design an appropriate number and complexity of barcodes for a sampling scheme.

### EXAMPLES

### Example 1: Clinical Trial Outcome Prediction for Novel Therapeutic Candidates: Genotype Specific Response

A bank is established using the methods described containing cancerous cells from thousands of patients with leukemia. A novel therapeutic candidate is applied to the cells at various doses and the relative growth rates of the genotype barcodes is measured with and without the application of the therapeutic. The ratio of these two numbers is used to determine if there is variation in therapeutic response (and therapeutic dose) associated with genotype.

This method may also be employed for existing therapeutics upon re-stratification for specific genotypes and/or other cellular biomarkers.

### Example 2: Clinical Trial Outcome Prediction for Novel Therapeutic Candidates: Genotype Specific Toxicity

A bank is established using the methods described containing normal fibroblast cells from thousands of healthy patients. The cells can be reprogrammed and differentiated in a pooled fashion into a cell type that would be sensitive to the therapeutic (ex: hepatocytes). A novel therapeutic candidate is applied to the cells at various doses and the expression level of biomarkers associated with toxicity is determined through single cell phenotypic assays such as RNA-seq, microscopy or flow cytometry. In the case of flow cytometry, cells are sorted based on toxicity markers. The presence of genotype barcodes in high toxicity bins is can be used to stratify patients for selection in a Phase I clinical trial.

This method may also be employed for existing therapeutics upon re-stratification for specific genotypes and/or other cellular biomarkers.

The methods described herein may also facilitate personalized dosing, e.g., in the treatment of a disease or condition using a therapeutic agent.

### Example 3: Clinical Trial Outcome Prediction for Novel Therapeutic Candidates: Genotype Specific Adjuvant Therapies

A bank is established using the methods described containing reprogrammed neurons from patients with Alzheimer's. A novel therapeutic candidate is applied to the cells. Additionally, a genetic screen is performed on the cells where the knockouts/knockdown/overexpression corresponding to the perturbations map to targeted therapeutics or gene therapies. Synergies between therapeutic response, genetic perturbation, and genotype are determined by single cell phenotypic assays such as RNA-seq, microscopy, or flow cytometry. For example, the expression level of alpha synuclein could be used as a biomarker of response.

This method may also be employed for existing therapeutics upon re-stratification for specific genotypes and/or other cellular biomarkers.

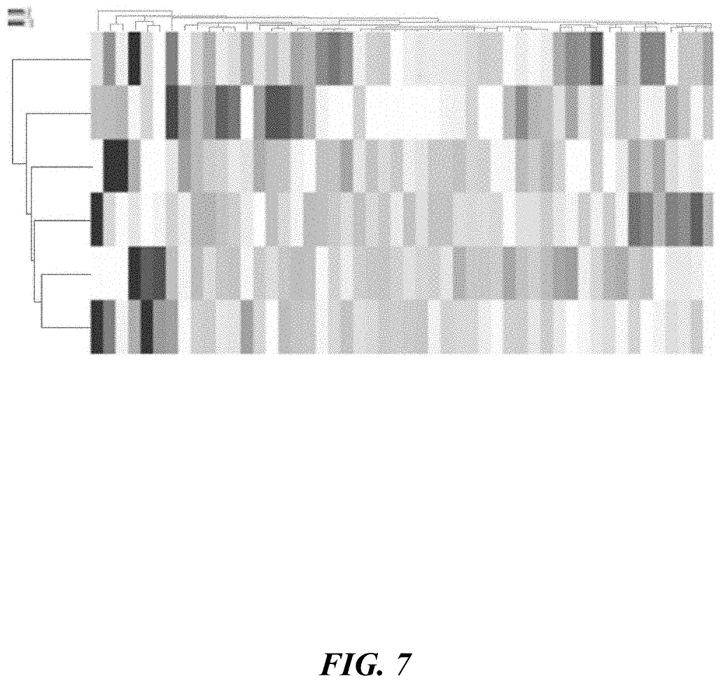

FIG. 7 shows gene expression signatures of patient cells subjected to a panel of drugs and conditions. The gene expression signatures are defined based on the average change from baseline associated with a treatment condition. A column corresponds to a different patient and a row corresponds to different treatment conditions. The top row corresponds to a condition in which cells are subject to a model of aging. The other rows correspond to treatment with Food and Drug Administration (FDA) approved drug compounds. The treatment conditions are Z-normalized across patients. The range of shading represent a six standard deviation dynamic range. This approach can be used to stratify patients for selecting optimal therapy using new biomarkers and new targets for drug discovery.

### Example 4: Novel Therapeutic Candidates

A bank is established using the methods described from reprogrammed stem cells from hair samples from random human population that includes significant variation with respect to gender, ethnic, age, and medical conditions. The cells are differentiated into a range of cell types (ex: cardiomyocytes, hematopoietic stem cells, gamma aminobutyric acid- ergic (GABAergic) neurons) and molecularly profiled using a single cell assay (e.g., RNA-seq, ATAC-seq, etc.). Genetic variants are associated with phenotypic variation. Candidates for genetic perturbation are predicted and tested on the cells to generate leads for therapeutics.

### Example 5: Agricultural Applications: Plants

A bank is established using the methods described from a population of genetically diverse protoplasts (generated through natural variation or mutagenesis). The photosynthetic activity of the cell is determined by measurement of the expression level of genes in the pathways. Genetic variants associated with phenotypic variation are determined, and candidates for genetic perturbation are predicted and tested on the cells. The best candidates proceed to be grown into adult plants.

### Example 6: Agricultural Applications: Animals

A bank is established using the methods described from a population of genetically diverse animals (generated through natural variation or mutagenesis). A metric associated with the cell is determined by measurement of the expression level of genes in the pathways. Genetic variants associated with phenotypic variation are determined, and candidates for genetic perturbation are predicted and tested on the cells. The best candidates proceed to be grown into adult animals with desired characteristics.

### Example 7: Analysis of Perturbations

A plurality of cells corresponding to a subject (e.g., a human or animal subject) is provided. The plurality of cells is perturbed to, for example, replace a gene or portion thereof with a diverse set of genotypes for this gene. The perturbation is associated with a first perturbation barcode. The cell is also provided a genotype barcode (e.g., as described elsewhere herein). The perturbed cells thus includes a first perturbation barcode associated with the perturbation of the cell as well as a genotype barcode specific to the cell. Cells are then subjected to a second perturbation and a second perturbation barcode may be provided to the cell. Twice perturbed cells include a first perturbation barcode, a second perturbation barcode, and a genotype barcode. Twice perturbed cells are proliferated to generate one or more duplicates of the twice perturbed cells. The twice perturbed cells are then subjected to sequencing using, for example, the single-cell sequencing and/or deconvolution approaches described elsewhere herein. In this manner, associations between different perturbations may be identified. In an example, the first perturbation alters genetic diversity associated with genes encoding G protein-coupled receptors.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-111. (canceled)

112. A method of analyzing a plurality of cells, comprising:
(a) providing a plurality of cells derived from cells of a plurality of subjects, wherein said plurality of cells comprise a plurality of nucleic acid molecules, and wherein said plurality of nucleic acid molecules comprise a plurality of barcode sequences;
(b) sequencing nucleic acid molecules derived from said plurality of nucleic acid molecules of said plurality of cells, thereby generating a plurality of sequencing reads corresponding to said plurality of nucleic acid molecules, wherein a portion of said plurality of sequencing reads comprise said plurality of barcode sequences;
(c) processing said plurality of sequencing reads, which plurality of sequencing reads comprises said plurality barcode sequences; and
(d) using a barcode sequence of said plurality of barcode sequences to associate a subset of said plurality of sequencing reads with a subject of said plurality of subjects,
wherein, prior to (b), said plurality of cells is generated upon proliferating said cells of said plurality of subjects in a bulk growth environment.

113. The method of claim 112, wherein a subset of said plurality of nucleic acid molecules comprises said plurality of barcode sequences.

114. The method of claim 112, wherein said plurality of barcode sequences is endogenous to said plurality of cells.

115. The method of claim 112, further comprising, prior to (a), incorporating said plurality of barcode sequences into said plurality of nucleic acid molecules of said plurality of cells.

116. The method of claim 115, wherein said plurality of barcode sequences is incorporated into said plurality of cells via transduction.

117. The method of claim 115, wherein said plurality of barcode sequences is incorporated into said plurality of cells using a viral vector, transfection, homologous recombinant integration, Agrobacterium mediated gene transfer, an antibody-conjugated oligonucleotide, or an episomal vector.

118. The method of claim 112, wherein said barcode sequence of said plurality of barcode sequences comprises from 1 base to 1000 bases.

119. The method of claim 112, wherein said plurality of subjects comprises a plurality of human subjects.

120. The method of claim 112, wherein identities of said plurality of subjects are encrypted or ambiguated.

121. The method of claim 112, wherein said plurality of cells is derived from a bodily fluid.

122. The method of claim 112, wherein said plurality of cells comprises skin cells or hair cells.

123. The method of claim 112, wherein proliferated cells of said plurality of cells are stratified by growth rate.

124. The method of claim 112, wherein at least a subset of said plurality of barcode sequences comprises a plurality of perturbation barcode sequences associated with a plurality of perturbations.

125. The method of claim 124, wherein said plurality of perturbations are selected from the group consisting of addition of a small molecule, a knockout, an antibody, cell-cell interactions, RNAi, an open reading frame (ORF), and Clustered Regularly Interspaced Short Palindromic Repeats (CRISPR) single guide ribonucleic acid (sgRNA).

126. The method of claim 124, wherein said plurality of perturbations comprise a variation in temperature or a variation in pH.

127. The method of claim 124, wherein said plurality of perturbations comprise introduction of mutated forms of genes.

128. The method of claim 112, wherein at least a subset of said plurality of barcode sequences are associated with a plurality of measurements selected from the group consisting of RNA-seq, ATAC-seq, in-situ sequencing, and cell morphology measurements.

129. The method of claim 112, further comprising:
(e) introducing a plurality of fluorescent probes to said plurality of cells;
(f) subjecting said plurality of cells to conditions sufficient to hybridize said plurality of fluorescent probes to said plurality of barcode sequences; and
(g) optically detecting said plurality of fluorescent probes hybridized to said plurality of barcode sequences in said plurality of cells.

130. The method of claim 112, further comprising, prior to (b), processing said plurality of nucleic acid molecules to generate said nucleic acid molecules, which nucleic acid molecules are subsequently sequenced.

131. The method of claim 130, wherein said processing comprises (i) generating copies of said plurality of nucleic acid molecules or (ii) recovering said plurality of nucleic acid molecules from said plurality of cells.
